(19) United States
(12) Patent Application Publication
(10) Pub. No.: US 2019/0060190 A1
(43) Pub. Date: Feb. 28, 2019

(54) Dental Products and Methods

(71) Applicant: TriNova Dental, Inc., West Jordan, UT (US)

(72) Inventors: Brody Hart, Salt Lake City, UT (US); Teemu Scarborough, Coeur d'Alene, ID (US)

(21) Appl. No.: 16/119,903

(22) Filed: Aug. 31, 2018

Related U.S. Application Data
(60) Provisional application No. 62/552,919, filed on Aug. 31, 2017.

(51) Int. Cl. A61K 8/25; A61K 8/21; A61K 8/19; A61K 8/33; A61Q 11/00; A61K 8/26; A61C 9/00; A61C 19/06

(52) U.S. Cl. CPC A61K 8/25; A61K 8/21; A61K 8/19; A61K 8/33; A61K 2800/48; A61K 8/26; A61C 9/0033; A61C 19/063; A61Q 11/00

(57) ABSTRACT

An aqueous high-viscosity gingival hemostatic retraction paste includes greater than 45% by weight kaolin clay, greater than 20% by weight aluminum chloride hexahydrate, and less than 25% by weight water. The paste may also include a thickening agent. The paste may include thickening agent in an amount of between approximately 0.25% and 1% by weight. The paste may also include a desensitizer. The paste may include desensitizer in an amount between approximately 0.5% and approximately 5% by weight. A compression cap for application to a prepared tooth during a gingival retraction procedure includes a body having a first end with an opening and defining a hollowed-out center extending into the body from the opening, wherein the compression cap comprises a wax compound. A method for applying desensitizer to a prepared tooth during a gingival retraction procedure includes applying a desensitizer compound to an inner surface of the compression cap.

### CROSS-REFERENCE TO RELATED APPLICATIONS

This application claims the benefit of U.S. Provisional Application No. 62/552,919, Filed Aug. 31, 2017, which is incorporated herein by reference for all it discloses.

### BACKGROUND OF THE INVENTION

### 1. Field of the Invention

The present invention relates to gingival retraction, and more particularly to a high-viscosity gingival retraction paste with improved functionality.

### 2. Background and Related Art

When dentists are preparing a tooth for a dental restoration such as a crown, bridge, veneer, inlay, or onlay, the dentist typically wishes to control bleeding and retract the gingival sulcus prior to creating a dental impression or digital scanning for use in creating the fixed dental prosthesis. In the past, dentists have used retraction cord to achieve the goals of controlling bleeding and retracting the gingival sulcus. This entailed tucking a woven string, often impregnated with a hemostatic agent like epinephrine, into the sulcus. The practice of packing string to retract the gingival tissue is technique-sensitive, potentially damaging to the periodontal ligament, and is painful to the patient, thus requiring multiple injections of local anesthetic.

Attempts have been made to address the problems inherent with retraction cord. Specifically, various aqueous or paste-type compounds have been created and utilized in attempts to achieve the goals of controlling bleeding and retracting the gingival sulcus. While such compounds have achieved varying degrees of success, certain problems remain. Some compounds lack adequate effectiveness or are slow to take effect, which may result in the dentist having to attempt to reapply the material or eventually to resort to retraction cord. Another problem with such methods is that it can be difficult using such methods to retain the compound in the sulcus where the compound is needed while simultaneously keeping saliva away from the compound to prevent dilution and loss of effectiveness of the compound. Some compounds are also difficult to apply to the desired area. If the compound is not applied or retained in the proper area, it may have adverse effects such as leaving a bad taste in the mouth of the patient.

Another problem with such methods is that it can be difficult using such methods to properly apply a desensitizer such as glutaraldehyde (sometimes sold under the brand name Gluma) to the prepared tooth to reduce the patient's sensitivity to further aspects of the procedure. A primary difficulty is that existing methods typically do not result in the desensitizer being applied for a sufficient time as to take effect. In sum, current gingival retraction methods leave much to be desired.

### BRIEF SUMMARY OF THE INVENTION

A clay-based gingival hemostatic retraction paste addresses many of the concerns with existing products and methods. The paste is aqueous and has a high viscosity compared with existing products. Additionally, the paste has a high concentration of aluminum chloride hexahydrate, providing a significantly improved astringent effect. Embodiments of the paste have proven significantly better at stopping bleeding in a short period of time when compared with existing products.

According to implementations of the invention, an aqueous high-viscosity gingival hemostatic retraction paste includes greater than 45% by weight kaolin clay, greater than 20% by weight aluminum chloride hexahydrate, and less than 25% by weight water. The paste may also include a thickening agent. The thickening agent may include any of xanthan gum, chia seed powder, arrowroot powder, flax seed powder, potato starch, tapioca starch, and carrageenan powder. The paste may include the thickening agent in an amount of between approximately 0.25% and 1% by weight.

The paste may also further include sodium fluoride as a desensitizer and anticarie agent. Additionally and alternatively, the paste may include potassium nitrate as a desensitizer. The paste may include between approximately 0.5% and approximately 2% potassium nitrate by weight. In one implementation, the paste may include approximately 1% potassium nitrate by weight.

The paste may include between approximately 48% and approximately 53% kaolin clay by weight. In certain implementations, the paste includes greater than 50% by weight kaolin clay. In certain implementations, the paste includes approximately 51% by weight kaolin clay.

The paste may include between approximately 22% and approximately 26% aluminum chloride hexahydrate by weight. In certain implementations, the paste includes greater than 24% by weight aluminum chloride hexahydrate.

The paste may include between approximately 18% and approximately 22% water by weight. In certain implementations, the paste includes approximately 20% by weight water. The water may be distilled water.

According to certain implementations of the invention, an aqueous high-viscosity gingival hemostatic retraction paste includes between approximately 48% and approximately 53% by weight kaolin clay, between approximately 22% and approximately 26% by weight aluminum chloride hexahydrate, between approximately 18% and approximately 22% by weight water, and a thickening agent. The thickening agent may include any of the agents mentioned above.

The paste may include a desensitizer such as between approximately 0.5% and approximately 2% by weight potassium nitrate, between approximately 1% and approximately 4% by weight hydroxyethyl methacrylate, between approximately 0.5% and approximately 2% by weight glutaraldehyde, and approximately 0.15% weight to volume sodium fluoride.

According to implementations of the invention, an aqueous high-viscosity gingival hemostatic retraction paste includes between approximately 48% and approximately 53% by weight kaolin clay, between approximately 22% and approximately 26% by weight aluminum chloride hexahydrate, between approximately 18% and approximately 22% by weight water, between approximately 0.5% and approximately 2% by weight potassium nitrate, and a thickening agent. The thickening agent may include any of the agents mentioned above.

According to further implementations of the invention, a method for making an aqueous high-viscosity gingival hemostatic retraction paste includes dissolving the following ingredients in between approximately 18% and approximately 22%, by weight of the final paste, water: between approximately 22% and approximately 26%, by weight of the final paste, aluminum chloride hexahydrate, between approximately 0.5% and approximately 2%, by weight of the final paste, potassium nitrate and a thickening agent. The method further includes, while stirring, slowly adding between approximately 48% and approximately 53%, by weight of the final paste, kaolin clay and whipping a resulting mixture until a viscous paste is achieved.

A compression cap and a method for using the compression cap in conjunction with a gingival retraction procedure further addresses many of the concerns with existing products and methods. The method is performed in conjunction with a gingival retraction procedure in such a way as to enhance effectiveness of the gingival retraction procedure.

In accordance with implementations of the invention, a compression cap for application to a prepared tooth during a gingival retraction procedure includes a body having a first end with an opening and defining a hollowed-out center extending into the body from the opening, wherein the compression cap includes a wax compound. The wax compound may be a viscous wax, and may be more easily moldable when warmed. The body may have a second end adapted to receive a biting force of the patient.

The compression cap may be adapted to be heated to soften the wax compound, molded such that the hollowed-out center conforms to a prepared tooth, and then cooled to harden the wax compound as thus molded. Molding the compression cap may include any or all of creating an impression of the prepared tooth, creating an impression of a sulcus surrounding the prepared tooth, ensuring that the compression cap inserts into the sulcus of the prepared tooth, and/or forming wings that extend beyond the sulcus of the prepared tooth.

The compression cap may be formed of a hydrophobic compound. The compression cap may initially be sized or selected of a size to approximately cover a prepared tooth.

According to further implementations of the invention, a compression cap for application to a prepared tooth during a gingival retraction procedure includes a body having a first end with an opening and defining a hollowed-out center extending into the body from the opening, wherein the compression cap includes a wax compound that is more easily moldable when warmed and that is effectively hard at temperatures near human body temperature. The wax compound may include a viscous wax, and may include a hydrophobic compound.

As discussed above, the body has a second end adapted to receive a biting force. The compression cap may be adapted to be heated to soften the wax compound, molded such that the hollowed-out center conforms to a prepared tooth, and cooled to harden the wax compound. The molding actions may include those discussed above.

According to implementations of the invention, a method for using a wax-based compression cap in a gingival retraction procedure includes selecting an appropriate size of a compression cap formed of a wax compound, warming the compression cap until the wax compound softens sufficiently to allow the compression cap to be molded to a prepared tooth, placing the compression cap over the prepared tooth, molding the compression cap over the prepared tooth. removing the compression cap from the prepared tooth, and cooling the compression cap until the wax compound hardens. The method may optionally further include rinsing and drying the prepared tooth and a sulcus surrounding the prepared tooth, applying a gingival retraction paste to the sulcus, placing the compression cap over the prepared tooth, the gingival retraction paste, and the sulcus in a proper orientation, and having a patient bite firmly on the compression cap for a time sufficient to achieve desired gingival retraction.

The method may optionally include applying a desensitizer to an inner surface of the compression cap. Molding the compression cap over the prepared tooth may include any or all of creating an impression of the prepared tooth, creating an impression of a sulcus surrounding the prepared tooth, ensuring that the compression cap inserts into the sulcus of the prepared tooth, and/or forming wings that extend beyond the sulcus of the prepared tooth. The compression cap so used may have any of the features discussed above.

A method for applying desensitizer to a prepared tooth in conjunction with gingival retraction further addresses many of the concerns with existing products and methods. The method is performed in conjunction with a gingival retraction procedure in such a way as to better perform the desensitization function without interfering with the gingival retraction procedure. Indeed, implementation of aspects of the method may simultaneously improve effectiveness of the gingival retraction procedure. Implementation of aspects of the method better ensure that a desensitizing compound such as glutaraldehyde is in contact with the prepared tooth for a recommended time for adequate effectiveness.

In accordance with implementations of the invention, a method for applying desensitizer to a prepared tooth during a gingival retraction procedure includes applying a desensitizer compound to an inner surface of a compression cap sized and adapted to engage a prepared tooth and applying the desensitizer compound to the prepared tooth. The method also includes applying a retraction paste to a sulcus of the prepared tooth, applying the compression cap to the prepared tooth such that the inner surface of the compression cap contacts the prepared tooth and such that the compression cap retains the retraction paste in the sulcus, and leaving the compression cap on the prepared tooth a period of time such that the desensitizer compound is effective in desensitizing the prepared tooth.

The compression cap may be formed of or include a viscous wax compound. In some instances, the wax compound is more easily moldable when warmed. Thus, the method may include warming the compression cap to soften the wax compound, molding the compression cap over the prepared tooth, and cooling the compression cap to harden the wax compound. Molding the compression cap over the prepared tooth may include ensuring that the compression cap inserts into the sulcus. Molding the compression cap over the prepared tooth may further include forming wings that extend beyond the sulcus. Molding the compression cap over the prepared tooth may include creating an impression of the prepared tooth and/or creating an impression of the sulcus.

The compression cap may be formed of or include a hydrophobic compound. The desensitizing compound may be or include an effective amount of glutaraldehyde.

The compression cap may be adapted to receive a biting force from a patient so as to cause the inner surface of the compression cap to engage the prepared tooth and further to cause the compression cap to compress the retraction paste in the sulcus. The retraction paste may be an aqueous high-viscosity gingival hemostatic retraction paste.

According to further implementation of the invention, a method for applying desensitizer to a prepared tooth during a gingival retraction procedure includes providing a compression cap formed of a viscous wax compound and generally sized to engage a prepared tooth, softening the wax compound of the compression cap, molding the compression cap over the prepared tooth, and hardening the wax compound of the compression cap. The method also includes applying a desensitizer compound to an inner surface of the compression cap, applying the desensitizer compound to the prepared tooth, applying a retraction paste to a sulcus of the prepared tooth, applying the compression cap to the prepared tooth such that the inner surface of the compression cap contacts the prepared tooth and such that the compression cap retains the retraction paste in the sulcus, and leaving the compression cap on the prepared tooth a period of time such that the desensitizer compound is effective in desensitizing the prepared tooth.

As described above, the wax compound may be more easily moldable when warmed. Molding the compression cap over the prepared tooth may include ensuring that the compression cap inserts into the sulcus, forming wings that extend beyond the sulcus, creating an impression of the prepared tooth, and/or creating an impression of the sulcus. Similarly, as discussed above, the desensitizing compound may be or include an effective amount of glutaraldehyde.

According to further implementation of the invention, a method for applying glutaraldehyde to a prepared tooth during a gingival retraction procedure includes providing a compression cap formed of a viscous wax compound and generally sized to engage a prepared tooth, softening the wax compound of the compression cap, molding the compression cap over the prepared tooth, and hardening the wax compound of the compression cap. The method also includes applying glutaraldehyde to an inner surface of the compression cap, applying glutaraldehyde to the prepared tooth, applying a retraction paste to a sulcus of the prepared tooth, applying the compression cap to the prepared tooth such that the inner surface of the compression cap contacts the prepared tooth and such that the compression cap retains the retraction paste in the sulcus with the glutaraldehyde on the inner surface in contact with the prepared tooth, and leaving the compression cap on the prepared tooth a period of time such that the glutaraldehyde is effective in desensitizing the prepared tooth.

### BRIEF DESCRIPTION OF THE SEVERAL VIEWS OF THE DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows a front view of an embodiment of a compression cap; and

FIG. 2 shows a side view of an embodiment of a compression cap.

### DETAILED DESCRIPTION OF THE INVENTION

A description of embodiments of the present invention will now be given with reference to the Figures. It is expected that the present invention may take many other forms and shapes, hence the following disclosure is intended to be illustrative and not limiting, and the scope of the invention should be determined by reference to the appended claims.

In the disclosure and claims, formulations of components may be described using certain percentages or fractions. In all instances, unless otherwise indicated, the percentages or fractions are by weight.

According to embodiments of the invention, an aqueous high-viscosity gingival hemostatic retraction paste includes greater than 45% by weight kaolin clay, greater than 20% by weight aluminum chloride hexahydrate, and less than 25% by weight water. The paste may also include a thickening agent. The thickening agent may include any of xanthan gum, chia seed powder, arrowroot powder, flax seed powder, potato starch, tapioca starch, and carrageenan powder. The paste may include the thickening agent in an amount of between approximately 0.25% and 1% by weight.

The paste may also further include sodium fluoride as a desensitizer and anticarie agent. Additionally and alternatively, the paste may include potassium nitrate as a desensitizer. The paste may include between approximately 0.5% and approximately 2% potassium nitrate by weight. In one embodiment, the paste may include approximately 1% potassium nitrate by weight.

The paste may include between approximately 48% and approximately 53% kaolin clay by weight. In certain embodiments, the paste includes greater than 50% by weight kaolin clay. In certain embodiments, the paste includes approximately 51% by weight kaolin clay.

The paste may include between approximately 22% and approximately 26% aluminum chloride hexahydrate by weight. In certain embodiments, the paste includes greater than 24% by weight aluminum chloride hexahydrate.

The paste may include between approximately 18% and approximately 22% water by weight. In certain embodiments, the paste includes approximately 20% by weight water. The water may be distilled water.

According to certain embodiments of the invention, an aqueous high-viscosity gingival hemostatic retraction paste includes between approximately 48% and approximately 53% by weight kaolin clay, between approximately 22% and approximately 26% by weight aluminum chloride hexahydrate, between approximately 18% and approximately 22% by weight water, and a thickening agent. The thickening agent may include any of the agents mentioned above.

The paste may include a desensitizer such as between approximately 0.5% and approximately 2% by weight potassium nitrate, between approximately 1% and approximately 4% by weight hydroxyethyl methacrylate, between approximately 0.5% and approximately 2% by weight glutaraldehyde, and approximately 0.15% weight to volume sodium fluoride.

According to embodiments of the invention, an aqueous high-viscosity gingival hemostatic retraction paste includes between approximately 48% and approximately 53% by weight kaolin clay, between approximately 22% and approximately 26% by weight aluminum chloride hexahydrate, between approximately 18% and approximately 22% by weight water, between approximately 0.5% and approximately 2% by weight potassium nitrate, and a thickening agent. The thickening agent may include any of the agents mentioned above.

According to further embodiments of the invention, a method for making an aqueous high-viscosity gingival hemostatic retraction paste includes dissolving the following ingredients in between approximately 18% and approximately 22%, by weight of the final paste, water: between approximately 22% and approximately 26%, by weight of the final paste, aluminum chloride hexahydrate, between approximately 0.5% and approximately 2%, by weight of the final paste, potassium nitrate and a thickening agent. The method further includes, while stirring, slowly adding between approximately 48% and approximately 53%, by weight of the final paste, kaolin clay and whipping a resulting mixture until a viscous paste is achieved.

Gingival retraction pastes in accordance with embodiments of the invention described herein provide significant benefits over gingival retraction cord and other gingival retraction products. Gingival retraction pastes in accordance with embodiments of the invention provide reduced time of effectiveness, improved control of bleeding, reduced adverse effects such as bad taste and pain, and provide increased ease of use for dental professionals.

In accordance with embodiments of the invention, a compression cap for application to a prepared tooth during a gingival retraction procedure includes a body having a first end with an opening and defining a hollowed-out center extending into the body from the opening, wherein the compression cap includes a wax compound. The wax compound may be a viscous wax, and may be more easily moldable when warmed. The body may have a second end adapted to receive a biting force of the patient. The compression cap may be adapted to receive a biting force from a patient so as to cause the inner surface of the compression cap to engage the prepared tooth and further to cause the compression cap to compress the retraction paste in the sulcus. The retraction paste may be an aqueous high-viscosity gingival hemostatic retraction paste, as is disclosed herein.

The compression cap may be adapted to be heated to soften the wax compound, molded such that the hollowed-out center conforms to a prepared tooth, and then cooled to harden the wax compound as thus molded. Molding the compression cap may include any or all of creating an impression of the prepared tooth, creating an impression of a sulcus surrounding the prepared tooth, ensuring that the compression cap inserts into the sulcus of the prepared tooth, and/or forming wings that extend beyond the sulcus of the prepared tooth.

The compression cap may be formed of a hydrophobic compound. The compression cap may initially be sized or selected of a size to approximately cover a prepared tooth.

According to further embodiments of the invention, a compression cap for application to a prepared tooth during a gingival retraction procedure includes a body having a first end with an opening and defining a hollowed-out center extending into the body from the opening, wherein the compression cap includes a wax compound that is more easily moldable when warmed and that is effectively hard at temperatures near human body temperature. The wax compound may include a viscous wax, and may include a hydrophobic compound.

As discussed above, the body has a second end adapted to receive a biting force. The compression cap may be adapted to be heated to soften the wax compound, molded such that the hollowed-out center conforms to a prepared tooth, and cooled to harden the wax compound. The molding actions may include those discussed above.

According to embodiments of the invention, a method for using a wax-based compression cap in a gingival retraction procedure includes selecting an appropriate size of a compression cap formed of a wax compound, warming the compression cap until the wax compound softens sufficiently to allow the compression cap to be molded to a prepared tooth, placing the compression cap over the prepared tooth, molding the compression cap over the prepared tooth. removing the compression cap from the prepared tooth, and cooling the compression cap until the wax compound hardens. The method may optionally further include rinsing and drying the prepared tooth and a sulcus surrounding the prepared tooth, applying a gingival retraction paste to the sulcus, placing the compression cap over the prepared tooth, the gingival retraction paste, and the sulcus in a proper orientation, and having a patient bite firmly on the compression cap for a time sufficient to achieve desired gingival retraction.

The method may optionally include applying a desensitizer to an inner surface of the compression cap. Such application may form a part of a method for applying a desensitizer to the prepared tooth, as is disclosed herein.

Molding the compression cap over the prepared tooth may include any or all of creating an impression of the prepared tooth, creating an impression of a sulcus surrounding the prepared tooth, ensuring that the compression cap inserts into the sulcus of the prepared tooth, and/or forming wings that extend beyond the sulcus of the prepared tooth. The compression cap so used may have any of the features discussed above.

In accordance with embodiments of the invention, a method for applying desensitizer to a prepared tooth during a gingival retraction procedure includes applying a desensitizer compound to an inner surface of a compression cap sized and adapted to engage a prepared tooth and applying the desensitizer compound to the prepared tooth. The method also includes applying a retraction paste to a sulcus of the prepared tooth, applying the compression cap to the prepared tooth such that the inner surface of the compression cap contacts the prepared tooth and such that the compression cap retains the retraction paste in the sulcus, and leaving the compression cap on the prepared tooth a period of time such that the desensitizer compound is effective in desensitizing the prepared tooth.

The compression cap may be formed of or include a viscous wax compound. In some instances, the wax compound is more easily moldable when warmed. Thus, the method may include warming the compression cap to soften the wax compound, molding the compression cap over the prepared tooth, and cooling the compression cap to harden the wax compound. Molding the compression cap over the prepared tooth may include ensuring that the compression cap inserts into the sulcus. Molding the compression cap over the prepared tooth may further include forming wings that extend beyond the sulcus. Molding the compression cap over the prepared tooth may include creating an impression of the prepared tooth and/or creating an impression of the sulcus.

The compression cap may be formed of or include a hydrophobic compound. The desensitizing compound may be or include an effective amount of glutaraldehyde.

The compression cap may be adapted to receive a biting force from a patient so as to cause the inner surface of the compression cap to engage the prepared tooth and further to cause the compression cap to compress the retraction paste in the sulcus. The retraction paste may be an aqueous high-viscosity gingival hemostatic retraction paste, as is disclosed herein. The compression cap may be of a type disclosed herein.

According to further embodiments of the invention, a method for applying desensitizer to a prepared tooth during a gingival retraction procedure includes providing a compression cap formed of a viscous wax compound and generally sized to engage a prepared tooth, softening the wax compound of the compression cap, molding the compression cap over the prepared tooth, and hardening the wax compound of the compression cap. The method also includes applying a desensitizer compound to an inner surface of the compression cap, applying the desensitizer compound to the prepared tooth, applying a retraction paste to a sulcus of the prepared tooth, applying the compression cap to the prepared tooth such that the inner surface of the compression cap contacts the prepared tooth and such that the compression cap retains the retraction paste in the sulcus, and leaving the compression cap on the prepared tooth a period of time such that the desensitizer compound is effective in desensitizing the prepared tooth.

As described above, the wax compound may be more easily moldable when warmed. Molding the compression cap over the prepared tooth may include ensuring that the compression cap inserts into the sulcus, forming wings that extend beyond the sulcus, creating an impression of the prepared tooth, and/or creating an impression of the sulcus. Similarly, as discussed above, the desensitizing compound may be or include an effective amount of glutaraldehyde.

According to further embodiments of the invention, a method for applying glutaraldehyde to a prepared tooth during a gingival retraction procedure includes providing a compression cap formed of a viscous wax compound and generally sized to engage a prepared tooth, softening the wax compound of the compression cap, molding the compression cap over the prepared tooth, and hardening the wax compound of the compression cap. The method also includes applying glutaraldehyde to an inner surface of the compression cap, applying glutaraldehyde to the prepared tooth, applying a retraction paste to a sulcus of the prepared tooth, applying the compression cap to the prepared tooth such that the inner surface of the compression cap contacts the prepared tooth and such that the compression cap retains the retraction paste in the sulcus with the glutaraldehyde on the inner surface in contact with the prepared tooth, and leaving the compression cap on the prepared tooth a period of time such that the glutaraldehyde is effective in desensitizing the prepared tooth.

Embodiments of the gingival retraction paste may be provided in a variety of forms and packaging. For example, the paste may be provided in single-unit dose cartridges that fit Centrix dental composite guns. Alternatively, the paste may be provided in a syringe with an applicator tip designed to allow placement of the paste into the sulcus. The high viscosity of the paste ensures that the paste remains in the location of application better than other products, improving effectiveness, and reducing the chance that the patient will taste the paste. The paste is able to absorb fluids (e.g. blood) at the sulcus, and in combination with the paste's astringent ingredients leads to consistent and rapid hemostasis. A variety of other benefits and features of the paste will be understood from the disclosure herein and from the use of the paste.

Application and effectiveness of the paste may be aided through use of a wax-based application cap, as disclosed herein. The compression cap as disclosed herein and its method for use are particularly useful in conjunction with a gingival retraction procedure. The compression cap serves both as a mechanism to retain a gingival retraction paste in the sulcus of the prepared tooth as well as a mechanism to simultaneously apply a desensitizer compound such as glutaraldehyde (sometimes sold under the product name Gluma) to the prepared tooth for a sufficient time as to allow the desensitizer compound to be adequately effective. Additionally, the compression cap and its method for use ensure that a gingival retraction paste is most effective in stopping bleeding and preparing the sulcus for further dental restoration work.

Methods in accordance with embodiments of the invention ensure that the retraction paste is located in the sulcus in a most effective manner. First, the compression cap may be molded to exactly fit the prepared tooth as well as the sulcus. Additionally, the dental practitioner can further mold the compression cap to create wings to better hold the retraction paste at the desired location of application.

Because the compression cap may be made of a hydrophobic material, such as a viscous wax compound, the compression cap serves as a barrier to prevent blood, saliva, or other bodily fluids from penetrating the compression cap and diluting or washing away the retraction paste. Further, because the compression cap may be exactly molded to the prepared tooth, there is little to no way for bodily fluids to pass around the compression cap and wash away the retraction paste.

The compression cap serves to press the retraction paste into the sulcus, forming a seal around the compression cap to better prevent entry of saliva under the compression cap, and also better stemming the flow of blood from the sulcus which might otherwise also wash away the retraction paste. All told, the embodiments of the methods described herein greatly improve the effectiveness of a gingival retraction treatment as part of a dental restoration procedure.

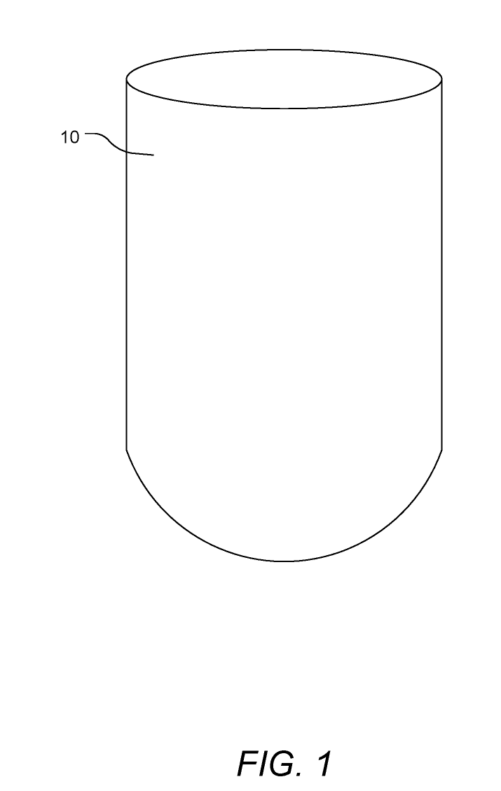

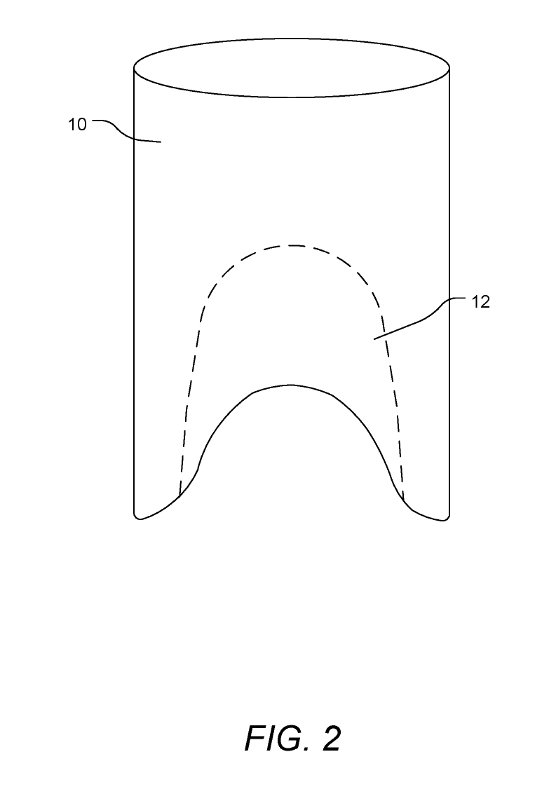

The embodiments of the compression caps are designed to improve placement and retention of the paste in the sulcus. By way of example, an exemplary compression cap 10 is illustrated in front and side views in FIGS. 1 and 2. The compression cap 10 may be provided in a variety of sizes to accommodate different sizes of teeth. For example, the compression cap may be roughly cylindrical in shape, as is illustrated in FIGS. 1 and 2, and may have an outer diameter of approximately 6 mm, approximately 9 mm, or approximately 12 mm, as appropriate. The dental professional could then select the size most appropriate for the tooth being treated. In one example, the compression cap 10, regardless of diameter, has a height of approximately 1.5 cm to allow the patient to bite down on the cap 10 to place pressure on the cap after placement. The cap 10 includes a hollowed-out center 12 on one end adapted to receive the tooth therein, as best illustrated in FIG. 2.

The compression cap may be made out of viscous wax and may come in various sizes. The wax is chosen to be generally solid at human body temperature, but generally moldable at a temperature that is above human body temperature but not so above human body temperature as to cause discomfort or burning when in contact with human skin or mouth parts. This cap is placed over the prepared tooth and the retraction material in order to force the retraction material apically into the sulcus, thus achieving improved retraction and hemostasis. The cap also keeps the retraction paste in the sulcus and does not allow fluids to interfere with the absorbent properties of the retraction paste. Other gingival retraction pastes come with a cotton compression cap. Cotton compression caps have certain limitations. First, cotton compression caps are not custom-fit to a specific sulcus. They generally come in three different sizes, and the dentist chooses the size that fits best. Inevitably, certain parts of the cotton compression cap do not insert into the gingival sulcus. Rather, cotton compression caps are too long and overlap the gingiva, or are too short and do not reach the sulcus. Thus, in that region of the sulcus, there is no downward pressure being applied. Second, cotton compression caps absorb oral fluids like saliva and blood. This results in the cotton cap becoming saturated and losing its compression ability. Third, cotton compression caps are limited in their ability to retain the retraction paste in the sulcus. Because fluids tend to saturate the cotton and creep into the sulcus, the gingival retraction paste also moistens and washes out of the sulcus into the oral cavity. The effectiveness of the paste is thus reduced and the patient experiences a very unpleasant taste.

While cotton compression caps may be used with embodiments of the gingival retraction paste, the compression cap in accordance with embodiments of the invention overcomes the deficiencies of cotton compression caps. The compression caps are custom fit to each gingival sulcus. The dentist molds the softened wax to the tooth preparation, creating an impression of the sulcus. The dentist also forms wax wings that extend slightly beyond the sulcus. These wings retain the retraction paste and form a barrier to saliva. The compression caps insert perfectly into the sulcus and apply even downward pressure. The compression caps do not absorb oral fluids. Wax is hydrophobic and thus forms a fluid barrier between the sulcus and the oral cavity. The gingival retraction paste remains unaffected by oral fluids within the compression cap once applied. The paste retains its effectiveness to absorb fluid and stop bleeding within the sulcus for the duration of the procedure. The compression caps retain the retraction paste in the sulcus. The gingival retraction paste is prevented from mixing with saliva. Thus, the effectiveness of the retraction paste is unhindered, and the patient does not taste the retraction paste.

The methods for applying desensitizer to a prepared tooth as disclosed herein are particularly useful in conjunction with a gingival retraction procedure. The compression cap serves both as a mechanism to retain a gingival retraction paste in the sulcus of the prepared tooth as well as a mechanism to simultaneously apply a desensitizer compound such as glutaraldehyde (sometimes sold under the product name Gluma) to the prepared tooth for a sufficient time as to allow the desensitizer compound to be adequately effective. Previous methods for applying desensitizer in conjunction with a dental restoration were less effective: the desensitizer compound was often washed away by blood, saliva, or other bodily fluids, such that the desensitizer compound often did not remain on the prepared tooth for the generally recommended length of time.

In contrast, methods in accordance with embodiments of the invention provide multiple mechanisms to ensure that the desensitizer compound is placed on and remains on the prepared tooth for an effective length of time. First, the compression cap may be molded to exactly fit the prepared tooth. When the desensitizer compound is then applied to the interior surface of the compression cap and the compression cap is replaced on the prepared tooth, the desensitizer compound is distributed to the entire prepared tooth. It is generally easier to ensure that the desensitizer compound is applied to the entire interior surface of the compression cap outside the patient's mouth than it is to ensure full coverage of the prepared tooth inside the patient's mouth.

That said, the method allows for increased redundancy by allowing a second application of the desensitizer compound directly to the prepared tooth after the compression cap is fitted but before the compression cap is applied again. If any spots of the prepared tooth are missed, the desensitizer compound applied to the compression cap will likely cover any missed spots. Similarly, if any spots of the interior surface of the compression cap are missed, such spots were likely covered on the prepared tooth itself.

Third, because the compression cap may be made of a hydrophobic material, such as a viscous wax compound, the compression cap serves as a barrier to prevent blood, saliva, or other bodily fluids from penetrating the compression cap and diluting or washing away the desensitizing compound. Further, because the compression cap may be exactly molded to the prepared tooth, there is little to no way for bodily fluids to pass around the compression cap and wash away the desensitizing compound.

Fifth, when the method is used with a highly viscous and effective gingival retraction paste and when the compression cap is adequately molded to the sulcus and/or prepared tooth, the compression cap serves to press the retraction paste into the sulcus, forming a seal around the compression cap to better prevent entry of saliva under the compression cap, and also better stemming the flow of blood from the sulcus which might otherwise also wash away the desensitizing compound. Finally, the desensitizing procedure is accomplished easily and simultaneously with a gingival retraction procedure. All told, the embodiments of the method described herein greatly improves the effectiveness of a gingival retraction treatment and of simultaneous application of desensitizing compound to the prepared tooth as part of a dental restoration procedure.

Gingival hemostatic retraction pastes in accordance with embodiments of the invention have a high viscosity which may be provided in part by a relatively low percentage of water, a relatively high percentage of kaolin clay, and/or by a thickening or binding agent, such as xanthan gum, chia seed powder, arrowroot powder, flax seed powder, potato starch, tapioca starch, or carrageenan powder. The high viscosity of the retraction paste serves to aid in placement and retention of the paste in the gingival sulcus. The high viscosity allows for controlled placement of desired amounts of the paste at desired locations in the sulcus and the high viscosity helps retain the paste where placed, thus reducing unwanted side effects such as tasting of the paste by the patient.

A paste in accordance with embodiments of the invention may include kaolin clay, which is a fine powder that acts as an absorbent, in relatively high amounts, such as in the range of approximately 48% to approximately 53% by weight of the final composition. In certain embodiments, the portion of the kaolin clay exceeds approximately 50% by weight. In one specific example, the paste includes approximately 51% kaolin clay by weight.

A paste in accordance with embodiments of the invention may include a relatively low amount of water (distilled water or other form of purified or food/medicine-grade water), such as less than approximately 25% water by weight. By way of example, formulations of the past may include between approximately 18% to approximately 22% distilled water by weight. In one specific example, the paste includes approximately 20% distilled water by weight.

A paste in accordance with embodiments of the invention may include a thickener, thickening agent, or binding agent. Examples of thickeners, thickening agents, or binding agents may include xanthan gum, chia seed powder, arrowroot powder, flax seed powder, potato starch, tapioca starch, or carrageenan powder. In one example, the formulation includes approximately 0.5% xanthan gum by weight. If another (or multiple) thickener, thickening agent, or binding agents are used with or in place of xanthan gum, they may be used in an amount effective to achieve a similar effect or to achieve a desired level of viscosity, as may be determined by routine experimentation.

Pastes in accordance with embodiments of the invention further include a relatively high amount of aluminum chloride hexahydrate, which acts as an astringent. For example, formulations in accordance with embodiments of the invention may include greater than approximately 20% by weight aluminum chloride hexahydrate. In certain embodiments, the paste includes between approximately 22% to approximately 26% aluminum chloride hexahydrate by weight. In one example, the paste includes approximately 24.5% aluminum chloride hexahydrate by weight.

In certain embodiments, the paste may include other ingredients, including ingredients to reduce sensitivity of the tooth being treated and for other purposes. The paste may include hydroxyethyl methacrylate, which acts to seal dentinal tubules to help control sensitivity, in an effective amount. For example, the composition may include between approximately 1% and approximately 4% hydroxyethyl methacrylate by weight. As a specific example, the composition includes approximately 2% hydroxyethyl methacrylate by weight.

The composition may include an effective amount of glutaraldehyde (sometimes sold under the brand name Gluma), which acts as a desensitizer and an antiseptic. For example, the paste may include between approximately 0.5% and approximately 2% glutaraldehyde by weight. In one specific example, the paste includes approximately 1% glutaraldehyde by weight.

The paste may include an effective amount of potassium nitrate, which acts as a desensitizer. By way of example, the composition may include between approximately 0.5% and approximately 2% potassium nitrate by weight. In one specific example, the composition includes approximately 1% potassium nitrate by weight.

The paste may also include an effective amount of an anticaries agent, such as sodium fluoride 0.15% weight per volume, which may also act as a desensitizer. The paste may also include effective amounts of flavoring agents (for those times when the paste is tasted by the patient) such as a mint flavoring agent. Similarly, the paste may include a coloring agent to help the paste achieve a desired color (such as to improve the appearance of the paste, or to improve visibility of placement of the paste). A non-limiting example of a coloring agent include FD&C (Federal Food, Drug, and Cosmetic Act) Yellow No. 5 (otherwise known as tartrazine or E102).

According to one embodiment of the invention, an exemplary clay-based gingival hemostatic retraction past has the following formulation (all percentages listed by weight): approximately 51% kaolin clay, approximately 24.5% aluminum chloride hexahydrate, approximately 20% distilled water, approximately 0.5% xanthan gum, approximately 2% hydroxyethyl methacrylate, approximately 1% glutaraldehyde, and approximately 1% potassium nitrate. Optionally, the composition may also include small but effective amounts of sodium fluoride and flavoring and coloring agents.

The paste may be prepared by dissolving all ingredients other than the kaolin clay into distilled water, e.g. by mixing. Then, while slowly stirring the mixture, kaolin clay is slowly added to the mixture until all clay is added. The mixture is then whipped until a viscous paste is achieved. The paste will be smooth and creamy with no particulates present.

Embodiments of the paste have numerous advantages over other existing retraction pastes. The high viscosity allows for good back-pressure during application to displace gingival sulcus. The paste is highly absorbent and highly astringent. The paste may also stay in the sulcus and not get diluted by saliva, thus retaining effectiveness and reducing the chances that the patient will taste the paste. The paste is antiseptic and desensitizing. The paste can be dispensed in a variety of manners, including in 0.5 mL cartridges compatible with Centrix dispensers.

The embodiments of the paste may be used in conjunction with compression caps designed to improve placement and retention of the paste in the sulcus, as described above.

In use, after preparing the tooth, the dental professional selects the appropriate size of compression cap and prepares it for the procedure by warming it until the wax becomes moldable or pliable, but not so warm as to completely lose all shape. This warming may be accomplished, for example, by placing the selected compression cap under warm to hot tap water for an appropriate time, such as 30 seconds. While the compression cap preferably comes pre-formed to include a hollow adapted to receive a prepared tooth, once the compression cap is moldable or pliable, it can be more specifically formed to the specific prepared tooth.

The compression cap is then molded over the prepared tooth, typically using the dentist's finger pressure. The dentist ensures that the compression cap inserts into the sulcus around the entire tooth and conforms to the prepared tooth. Additionally, the dentist forms wax wings that extend slightly beyond the sulcus. Optionally, the dentist may make a mark on or deform the compression cap so as to be able to easily reorient the compression cap. The dentist then removes the compression cap from the tooth and cools, such as by placing it in cold tap water for ten seconds.

Next, the dentist rinses and dries the prepared tooth. A desensitizer such as glutaraldehyde may be applied to the tooth and to the inside of the compression cap. The sulcus is dried as necessary to remove any bleeding, then the paste is applied to the sulcus, such as using a composite gun, using as much back pressure as possible as the material flows out. The compression cap is applied over the tooth, oriented correctly, placing pressure apically to better force the paste into the sulcus. Any excess paste may be removed, then the patient bites firmly on the compression cap for two minutes (e.g. the period of time recommended for the desensitizer to be effective). The compression cap is removed, the sulcus is rinsed and dried, after which the sulcus should be retracted and dry with margins of preparation clearly visible. If bleeding persists, the steps may be repeated from the steps of drying the sulcus and applying the paste. Once the desired effect is achieved, the restoration treatment may proceed with an impression or digital scan as with prior methods.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. An aqueous high-viscosity gingival hemostatic retraction paste comprising:
greater than 45% by weight kaolin clay;
greater than 20% by weight aluminum chloride hexahydrate; and
less than 25% by weight water.

2. An aqueous high-viscosity gingival hemostatic retraction paste as recited in claim 1, further comprising:
a thickening agent.

3. An aqueous high-viscosity gingival hemostatic retraction paste as recited in claim 2, wherein the thickening agent comprises an agent selected from the group consisting of:
xanthan gum;
chia seed powder;
arrowroot powder;
flax seed powder;
potato starch;
tapioca starch; and
carrageenan powder.

4. An aqueous high-viscosity gingival hemostatic retraction paste as recited in claim 2, wherein the paste comprises the thickening agent in an amount of between approximately 0.25% and 1% by weight.

5. An aqueous high-viscosity gingival hemostatic retraction paste as recited in claim 1, further comprising sodium fluoride as a desensitizer and anticarie agent.

6. An aqueous high-viscosity gingival hemostatic retraction paste as recited in claim 5, further comprising potassium nitrate as a desensitizer.

7. An aqueous high-viscosity gingival hemostatic retraction paste as recited in claim 6, wherein the paste comprises between approximately 0.5% and approximately 2% potassium nitrate by weight.

8. An aqueous high-viscosity gingival hemostatic retraction paste as recited in claim 6, wherein the paste comprises approximately 1% potassium nitrate by weight.

9. An aqueous high-viscosity gingival hemostatic retraction paste as recited in claim 1, wherein the paste comprises between approximately 48% and approximately 53% kaolin clay by weight.

10. An aqueous high-viscosity gingival hemostatic retraction paste as recited in claim 1, wherein the paste comprises greater than 50% by weight kaolin clay.

11. An aqueous high-viscosity gingival hemostatic retraction paste as recited in claim 1, wherein the paste comprises between approximately 22% and approximately 26% aluminum chloride hexahydrate by weight.

12. An aqueous high-viscosity gingival hemostatic retraction paste as recited in claim 1, wherein the paste comprises greater than 24% by weight aluminum chloride hexahydrate.

13. An aqueous high-viscosity gingival hemostatic retraction paste as recited in claim 1, wherein the paste comprises between approximately 18% and approximately 22% water by weight.

14. An aqueous high-viscosity gingival hemostatic retraction paste as recited in claim 1, wherein the paste comprises approximately 20% by weight water.

15. A compression cap for application to a prepared tooth during a gingival retraction procedure, the compression cap comprising:
a body having a first end with an opening and defining a hollowed-out center extending into the body from the opening;
wherein the compression cap comprises a wax compound.

16. A compression cap as recited in claim 15, wherein the compression cap is adapted to be heated to soften the wax compound, molded such that the hollowed-out center conforms to a prepared tooth, and cooled to harden the wax compound.

17. A compression cap as recited in claim 16, wherein molding the compression cap comprises an action selected from the group consisting of:
creating an impression of the prepared tooth;
creating an impression of a sulcus surrounding the prepared tooth;
ensuring that the compression cap inserts into the sulcus of the prepared tooth; and
forming wings that extend beyond the sulcus of the prepared tooth.

18. A method for applying desensitizer to a prepared tooth during a gingival retraction procedure, the method comprising:
applying a desensitizer compound to an inner surface of a compression cap sized and adapted to engage a prepared tooth;
applying the desensitizer compound to the prepared tooth;
applying a retraction paste to a sulcus of the prepared tooth;
applying the compression cap to the prepared tooth such that the inner surface of the compression cap contacts the prepared tooth and such that the compression cap retains the retraction paste in the sulcus; and
leaving the compression cap on the prepared tooth a period of time such that the desensitizer compound is effective in desensitizing the prepared tooth.

19. A method as recited in claim 18, wherein the desensitizing compound comprises an effective amount of glutaraldehyde.

20. A method as recited in claim 18, wherein the retraction paste is an aqueous high-viscosity gingival hemostatic retraction paste.
